(19) United States
(12) Patent Application Publication
(10) Pub. No.: US 2012/0177632 A1
(43) Pub. Date: Jul. 12, 2012

(54) METHODS OF OPTIMIZING DISEASE TREATMENT

(76) Inventors: Mari L. Shinohara, Durham, NC (US); Makoto Inoue, Durham, NC (US)

(21) Appl. No.: 13/347,233

(22) Filed: Jan. 10, 2012

Related U.S. Application Data
(60) Provisional application No. 61/431,127, filed on Jan. 10, 2011.

(51) Int. Cl. A61K 31/4025; G01N 33/573; A61K 31/7105; C12Q 1/37; A61K 39/395; A61K 31/713

(52) U.S. Cl. 424/130.1; 514/422; 514/44 A; 514/44 R; 435/23; 435/7.4

(57) ABSTRACT

Provided are methods for optimizing treatment of an autoimmune disease in a subject, methods for identifying and/or selecting a compound as a therapeutic for an autoimmune disease, methods of identifying a patient that is responsive to IFN-β therapy, and methods for identifying an agent that inhibits NLRP3 inflammasome activity.

### CROSS-REFERENCE TO RELATED APPLICATIONS

This patent application claims priority to U.S. Provisional Patent Application No. 61/431,127 filed Jan. 10, 2011, the entire content of which is incorporated herein by reference.

### FIELD OF THE DISCLOSURE

The disclosure relates to methods for treating an autoimmune disease, methods for identifying a patient that is responsive or non-responsive to an autoimmune disease therapy, and methods for identifying therapeutic compounds for treating autoimmune disease.

### BACKGROUND

Multiple sclerosis (“MS”) is an autoimmune disease in which myelin-reactive CD4⁺ T cells infiltrate the central nervous system (“CNS”) and induce demyelinating disease in the brain and spinal cord. The progressive demyelination associated with multiple sclerosis compromises neural signaling and may lead to irreversible nerve damage and severe disability.

Drugs comprising interferon-β (“IFN-β”, also referred to as “IFNβ”) provided the first disease-modifying therapies licensed for treating MS and have been widely used to treat MS for over 15 years. In the clinic today, IFN-β represents the first-line therapy for MS patients and remains the most common MS treatment. Drugs comprising IFN-β reduce MS symptoms, mitigate CNS damage, and slow the clinical progression of MS over time.

Though its clinical benefits are now well established, the precise mechanism by which IFN-β mitigates MS disease has been subject to debate. IFN-β is a pleiotropic, natural polypeptide product of the immune system with demonstrated activity in a broad and complex network of biological pathways. IFN-β acts through binding interactions with its cell-surface receptor IFNAR, stimulating expression of many genes with functional effects that vary from well characterized to largely unknown. Functional expression products from the various interferon-stimulated genes, whether alone or in coordination, mediate the numerous antiviral, growth-inhibitory, and immunoregulatory activities that have been attributed to IFN-β. Particularly noteworthy in the context of MS are several reported anti-inflammatory functions associated with IFN-β, but it remains unclear which among the myriad interferon-stimulated gene(s) and pathway(s) are responsible for the therapeutic effects observed in many MS patients receiving the drug.

Unfortunately, drugs comprising IFN-β have several disadvantages. For one, drugs comprising IFN-β are expensive. In addition, as might be expected with such a broadly active molecule, drugs comprising IFN-β exhibit many serious side effects, including flu-like symptoms, injection site reactions, myalgia, depression, liver damage, anemia, leukopenia, and thrombocytopenia. These toxic side effects impose a considerable burden, and many MS patients are unable to tolerate IFN-β treatment.

In addition, approximately one-third of MS patients turn out to be “non-responders” that experience no clinical benefit from IFN-β, but clinicians currently lack any reliable means to predict the response to IFN-β therapy in any given MS patient. Thus, most non-responders receive ineffective IFN-β treatments for 1-2 years until non-responsiveness can be empirically recognized. This lack of a prospective test imposes high socioeconomic costs, for non-responder MS patients fruitlessly suffer the side effects and expense of IFN-β treatment during a critical window for early treatment of a progressive degenerative disease.

### SUMMARY

In an aspect, the disclosure provides a method for identifying a subject or a patient class. In various embodiments of this aspect, the method comprises determining an expression level of a marker in a sample obtained from the subject or patient, and identifying the subject or the patient as a member of the subject or patient class based on the expression level of the marker in the sample relative to a control expression level of the marker. In some embodiments the subject or patient is identified as a member of the class when the expression level of the marker in the sample is increased relative to a control expression level of the marker. In some embodiments the subject or patient is identified as a non-member of the class when the expression level of the marker in the sample is not increased, or is decreased, relative to a control expression level of the marker.

In some embodiments of the aspect, the method can be used for optimizing a treatment regimen for an autoimmune disease in a patient and comprises obtaining a sample from the patient, determining an expression level of a marker in the sample, and adjusting the treatment regimen for the autoimmune disease in the patient to include or exclude a drug wherein the treatment regimen includes the drug when the expression level of the marker in the sample is increased relative to a control expression level and omits the drug when the expression level of the marker in the sample is not increased relative to a control expression level. The marker may indicate NLRP3 inflammasome activity. In some embodiments, the marker may be IL-1β and/or IL-18. In other embodiments, the marker may be caspase-1. The drug may comprise an inhibitor of NLRP3 inflammasome activity, and, in some embodiments, the drug may comprise IFN-β. The autoimmune disease may be multiple sclerosis. In some embodiments, the sample may comprise blood. And in some embodiments, the sample may comprise cerebrospinal fluid.

In some embodiments, the method described above for optimizing a treatment regimen for an autoimmune disease may further comprise processing the sample to substantially purify a cell type in the sample. Furthermore, the cell type may be a monocyte, a macrophage, a dendritic cell, or microglia in some embodiments.

In some embodiments, the method described above for optimizing a treatment regimen for an autoimmune disease may further comprise reviewing the medical history of the patient for the presence of microbial infections and/or optic neuritis. In some embodiments, the method described above for optimizing a treatment regimen for an autoimmune disease may further comprise measuring T cell infiltration in the brain and spinal cord.

In some embodiments of the aspect, the method can be used for selecting a drug for treating an autoimmune disease in a patient and comprises obtaining a sample from the patient, determining an expression level of a marker in the sample, and selecting the drug for treating the autoimmune disease in the patient when the expression level of the marker in the sample is increased relative to a control expression level. The marker may indicate NLRP3 inflammasome activity. In some embodiments, the marker may be IL-1β and/or IL-18. In other embodiments, the marker may be caspase-1. The drug may comprise an inhibitor of NLRP3 inflammasome activity. In some embodiments, the drug may comprise interferon-β. The autoimmune disease may be multiple sclerosis. In some embodiments, the sample may comprise blood. And in some embodiments, the sample may comprise cerebrospinal fluid.

In some embodiments, the method described above for selecting a drug for treating an autoimmune disease in a patient may further comprise processing the sample to substantially purify a cell type in the sample. Furthermore, the cell type may be a monocyte in some embodiments.

In some embodiments, the method described above for selecting a drug for treating an autoimmune disease in a patient may further comprise reviewing the medical history of the patient for the presence of microbial infections and/or optic neuritis. In some embodiments, the method described above for selecting a drug for treating an autoimmune disease in a patient may further comprise measuring T cell infiltration in the brain and spinal cord.

In some embodiments of the aspect, the method can be used for predicting efficacy of a drug for treating an autoimmune disease in a patient and comprises obtaining a sample from a patient and determining an expression level of a marker in the sample, wherein the method predicts high efficacy of the drug when the expression level of the marker in the sample is increased relative to a control expression level and wherein the method predicts low efficacy of the drug when the expression level of the marker in the sample is not increased relative to a control expression level. The marker may indicate NLRP3 inflammasome activity. In some embodiments, the marker may be IL-1β and/or IL-18. In other embodiments, the marker may be caspase-1. The drug may comprise an inhibitor of NLRP3 inflammasome activity. In some embodiments, the drug may comprise IFN-β. The autoimmune disease may be multiple sclerosis. In some embodiments, the sample may comprise blood. And in some embodiments, the sample may comprise cerebrospinal fluid.

In some embodiments, the method described above for predicting efficacy of a drug for treating an autoimmune disease in a patient may further comprise processing the sample to substantially purify a cell type in the sample. Furthermore, in some embodiments, the cell type may be a monocyte, a macrophage, a dendritic cell, or microglia.

In some embodiments, the method described above for predicting efficacy of a drug for treating an autoimmune disease in a patient may further comprise reviewing the medical history of the patient for the presence of microbial infections and/or optic neuritis. In some embodiments, the method described above for predicting efficacy of a drug for treating an autoimmune disease in a patient may further comprise measuring T cell infiltration in the brain and spinal cord.

In some embodiments of the aspect, the method can be used for identifying a patient with an autoimmune disease as non-responsive to treatment with a drug and comprises obtaining a sample from the patient and determining an expression level of a marker in the sample, wherein the patient is identified as non-responsive when the expression level of the marker in the sample is not increased relative to a control expression level. The maker may indicate NLRP3 inflammasome activity. In some embodiments, the marker may be IL-1β and/or IL-18. In other embodiments, the marker may be caspase-1. The drug may comprise an inhibitor of NLRP3 inflammasome activity. In some embodiments, the drug may comprise IFN-β. The autoimmune disease may be multiple sclerosis. In some embodiments, the sample may comprise blood. And in some embodiments, the sample may comprise cerebrospinal fluid.

In some embodiments, the method described above for identifying a patient with an autoimmune disease as non-responsive to treatment with a drug may further comprise processing the sample to substantially purify a cell type in the sample. Furthermore, the cell type may be a monocyte, a macrophage, a dendritic cell, or microglia in some embodiments.

In some embodiments, the method described above for identifying a patient with an autoimmune disease as non-responsive to treatment with a drug may further comprise reviewing the medical history of the patient for the presence of microbial infections and/or optic neuritis. In some embodiments, the method described above for identifying a patient with an autoimmune disease as non-responsive to treatment with a drug may further comprise measuring T cell infiltration in the brain and spinal cord.

In another aspect, the disclosure provides a method for identifying a compound capable of inhibiting NLRP3 inflammasome activity comprising contacting a cell with a test compound, contacting the cell with an activator of NLRP3, and determining whether the test compound inhibits NLRP3 activity in the cell. The activator of NLRP3 may comprise ATP. Furthermore, the cell may be a macrophage, a dendritic cell, or microglia. In some embodiments, the determining step may comprise measuring expression of IL-1β and/or IL-18. In other embodiments, the determining step may comprise measuring expression of caspase-1.

Another aspect of the disclosure provides a method for identifying a therapeutic compound for treating an NLRP3 inflammasome-independent autoimmune disease in a subject comprising administering a test compound to the patient and determining whether the autoimmune disease in the patient responds to the test compound. The subject may be a mammal, and in some embodiments, the mammal may be a human. Where the subject is a human, the NLRP3 inflammasome-independent autoimmune disease may be multiple sclerosis. In some embodiments, the mammal may be a mouse. Where the subject is a mouse, the mouse may comprise a condition or disease that is a model for a human autoimmune disease and, in some embodiments, the condition or disease may be experimental autoimmune encephalomyelitis. Furthermore, the mouse may lack a component of the NLRP3 inflammasome. In some embodiments, the missing component of the NLRP3 inflammasome may be NLRP3. And in some embodiments, the missing component of the NLRP3 inflammasome may be ASC or caspase-1.

In an aspect, the disclosure provides a method for treating a subject having NLRP3 inflammasome-independent autoimmune disease comprising administering to the subject an effective amount of a CCR2 inhibitor. In some embodiments, the subject may be a mammal such as, for example a mouse or a human. Where the subject is a human, the NLRP3 inflammasome-independent autoimmune disease may be multiple sclerosis.

In another aspect, the disclosure provides a method for identifying a subject having an increased risk of having or developing NLRP3 inflammasome-independent autoimmune disease, wherein the method comprises detecting T-cell infiltration in the central nervous system (CNS), wherein the subject is identified as having an increased risk of having or developing NLRP3 inflammasome-independent autoimmune disease when an amount of T-cell infiltration is detected in the CNS.

In another aspect, the disclosure provides a method for identifying a patient having an increased risk of having or developing NLRP3 inflammasome-independent autoimmune disease, wherein the method comprises determining whether the patient has, or is at risk of developing, optic neuritis, wherein the patient is determined to have or be at risk of developing optic neuritis, the patient is identified as having an increased risk of having or developing NLRP3 inflammasome-independent autoimmune disease.

In another aspect, the disclosure provides a method for identifying a patient having an increased risk of having or developing NLRP3 inflammasome-independent autoimmune disease, wherein the method comprises determining whether the patient has or has had a microbial infection, wherein the patient is determined to have or have had a microbial infection, the patient is identified as having an increased risk of having or developing NLRP3 inflammasome-independent autoimmune disease.

In another aspect, the disclosure provides a kit for performing any of the methods disclosed herein comprising at least one oligonucleotide primer for amplifying a marker and a buffer. The oligonucleotide primer is suitable for determining the expression levels of the marker in a biological sample from a subject. In some embodiments, the marker may be IL-1β and/or IL-18.

In another aspect, the disclosure provides a kit for performing any of the methods disclosed herein comprising at least one antibody that binds to a marker and a buffer. The antibody is suitable for determining the expression levels of the marker in a biological sample from a subject. In some embodiments, the marker may be IL-1β and/or IL-18.

The disclosure also provides for other aspects and embodiments that will be apparent to one of skill in the art in light of the following description.

### BRIEF DESCRIPTION OF THE DRAWINGS

FIG. 1 shows that IFNAR signaling suppresses IL-1β production through inhibition of NLRP3 inflammasome. (a)-(d) IL-1β protein expression in the supernatant of bone marrow-derived DC culture. (a) WT and Ifnar1^{-/-} DCs incubated with or without LPS for 48 h *p<0.05. (b) WT and Ifnar1^{-/-} peritoneal macrophages incubated with Ultrapure LPS alone (100 ng/ml), ATP alone (5 mM), or a combination of Ultrapure LPS and ATP for 24 h. (c) WT and Ifnar1^{-/-} peritoneal macrophages incubated with nigericin alone (2.5 μM) or combination of Ultrapure LPS and nigericin for 24 h. (d) Effect of 24 h pre-treatment of WT bone marrow-derived macrophages (BMMs) with recombinant IFNα (1,000 units/ml). (e, f) IFNAR signaling does not affect pro-IL-1β production. Expression of IL1β mRNA (e) and pro-IL-1β protein (f). (g-i) IFNAR signaling inhibits NLRP3 inflammasome. (g) Ifnar1^{-/-} macrophages showing elevated levels of active caspase-1 (Casp-1 p20). (h) Confocal microscopic images of caspase-1 (shown with arrows). Scale bars represent 10 μm. (i) Frequency of caspase-1 foci and integrated fluorescence intensity of caspase-1 foci. Data are presented as mean=SEM. *p<0.05.

FIG. 2 shows that IFNAR signaling suppresses in vitro activity of NLRP3 inflammasome. (a, b) IFNAR signaling inhibits ATP-induced ROS production. (a) ROS production (left panels). Numbers on the histograms denote DHE-positive cells. (b) Pretreatment (24 h) of WT BMM cells with rIFNα (1,000 units/ml) prior to incubation with ATP. (c-j) IFNAR signaling inhibits NADPH oxidase activity through inhibitory effect of SOCS-1. (c) Confocal microscopic images of p47^{phox} (a NADPH oxidase component; stain is shown with white). (d) Frequency of cell with p47^{phox} foci and average numbers of p47^{phox} foci per cell with or without rIFNβ treatment. (e) Reduction of Vav1 (guanine-nucleotide exchange factor for Rac) expression by rIFNβ treatment. (f) Socs1 mediates suppression of Vav1 expression by IFNβ. (g) Socs1 mRNA expression by qPCR. (h) rIFNα and rIFNβ inhibit activation of GTP Rac1, detected as GTP-bound Rac1 (Rac1-GTP). (i) SOCS1 mediates suppression of GTP activation by IFNβ. (j) SOCS1 associates with Rac1-GTP. (k, l) SOCS1 mediates inhibition of ROS (k) and IL-1β (l) production by IFNβ. (m) Schematic model for IFNα/β-mediated inhibition of the NLRP3 inflammasome through SOCS1.

FIG. 3 shows that IFNAR signaling suppresses in vivo activity of the NLRP3 inflammasome, which induces EAE progression. (a-d) NLRP3 inflammasome induces EAE progression. (a) EAE development in Asc^{-/-}, Nlrp3^{-/-}, and WT mice. (b, c) H&E staining (b) and LFB staining (c) of spinal cord sections from WT and Asc^{-/-} mice 17 days after EAE induction. Numbered squares indicate representative regions of cell infiltration (3(b) H&E staining shown as black dots) and demyelination (3(c) LFB staining shown as the loss of gray staining in WT) shown at high magnification. Arrowheads indicate regions of demyelination. (d) Numbers of total leukocytes and CD4⁺ T cells in spinal cords and brains of WT, Asc^{-/-}, and Nlrp3^{-/-} mice 17 days after EAE induction. Horizontal lines denote mean values. (e-h) NLRP3-inflammasome is activated during EAE progression. (e, f) Serum IL-1β (e) and IL-18 (f) levels in WT, Asc^{-/-}, and Nlrp3^{-/-} mice on the indicated days after EAE induction. (g) IL-1β production in splenocytes isolated from WT and Asc^{-/-} mice nine days after EAE induction. (h) Detection of active caspase-1 (Casp-1 p20) signaling in splenocytes isolated from WT, Asc^{-/-}, and Nlrp3^{-/-} mice nine days after EAE induction. (i-k) IFNAR signaling suppresses NLRP3-inflammasome activity in vivo. (i) Serum IL-1β levels in WT mice 9 days after EAE induction. Left panel shows comparison of serum IL-1β levels in mice with or without IFNβ-1b treatment. Right panel shows comparison of serum IL-1β levels between WT and Ifnar1^{-/-} mice. (j) IL-1β production by LPS-stimulated splenocytes isolated from mice 9 days after EAE induction. Left panel shows comparison between WT mice treated with or without IFNβ-1b. Right panel shows IL-1β production by splenocytes isolated from WT and Ifnar1^{-/-} mice nine days after EAE induction. (k) Active caspase-1 (Casp-1 p20) detection in splenocytes isolated from WT mice with or without IFNβ-1b treatment (left panels), and from WT or Ifnar4^{-/-} mice (right panels).

FIG. 4 shows that IFNβ-1b ameliorates EAE only when the progression is NLRP3 inflammasome-mediated. (a) EAE development in WT mice with or without IFNβ-1b treatment. (b) EAE severity was evaluated by the area under the curve (AUC) from time course data shown in (a). (c) Severity of EAE in WT, Asc^{-/-}, and Nlrp3^{-/-} mice with titrated dosages of Mtb used for immunization. (d) IFNβ-1b ameliorates EAE in WT mice induced by condition of Mtb use (left panel), but EAE was not ameliorated in Nlrp3^{-/-} (middle panel) and Asc^{-/-} mice (right panel). n=9-12. *; p<0.05. N.S.=not significant.

FIG. 5 shows that IFNAR signaling suppresses NLRP3 inflammasome activity. (a-c) IL-1β protein expression in culture supernatants of peritoneal macrophages—(a) Ultrapure LPS plus ATP; (b) nigericin; and BMMs (c) with MSU. (d) IL-18 protein expression in culture supernatant of BMMs. (e) ELISA analysis of expression of TNFα in peritoneal macrophage culture supernatants. Data are presented as mean±SEM. *p<0.05.

FIG. 6 shows IFNAR signaling does not affect expression of NLRP3 inflammasome components, P2X7R, and CD39, but induces lysosomal stabilization. (a) Evaluation of Nlrp3, Asc, Casp1 and Txnip mRNA expression by qPCR. (b) Cell surface expression of ATP receptor P2X7R and CD39 (nucleoside triphosphate diphosphohydrolase 1). (c) IFNAR signaling suppresses Alum-induced lysosomal rupture. Representative confocal microscopy images of DQ ovalbumin staining (left). DQ ovalbumin staining shown with white in the figure locates lysosomes. Quantitative evaluation of cells with DQ ovalbumin leakage to cytoplasm due to ruptured lysosomes. The middle panel shows comparison between WT and Ifnar1^{-/-} cells; right panel shows WT cells treated with rIFNα. (d) Evaluation of pg91^{phox} mRNA expression in WT BMMs by qPCR.

FIG. 7 depicts indication of IFNβ-1b activation to mouse cell. Evaluating induction of Socs-1, Ip10 and Mx1 mRNA expression by IFNβ treatment in WT BMMs.

FIG. 8 shows NLRP3-independent EAE development in WT mice with or without IFNβ-1b treatment.

FIG. 9 shows passive EAE development requires the NLRP3 inflammasome in recipients, and can be treated with IFNβ therapy. (A) EAE was induced by adoptively transfer CD4⁺ T cells from WT mice, which developed EAE, into Rag2^{-/-} (0; no T/B cells) or Nlrp^{-/-}3 Rag2^{-/-} (▴; no T/B cells, no NLRP3 inflammasome) recipient mice. Another group of Rag2^{-/-} had IFNβ treatment (). (B) and (C) Evaluation of the NLRP3 inflammasome activation status by (B) IL-1b and (C) activated caspase-1 (p20) by Western blot.

FIG. 10 shows CD4⁺ T cell infiltration into the CNS. CD4⁺ T cell numbers were analyzed in mice at the peak of EAE (for mice that develop EAE) in brain (A) and spinal cord (B). Horizontal bars show mean values.

FIG. 11 shows CCR2 deficient mice are resistant to EAE by aggressive disease induction. NLRP3-independent EAE was induced by an aggressive regimen, described in Example 5, in WT (◯) and Ccr2^{-/-} mice ().

FIG. 12 shows that IFNα and IFNβ inhibit ROS generation by mitochondria. (A) Time course of ATP-induced mitochondrial ROS generation in macrophages. (B) Suppression of mitochondrial ROS generation by type-1 IFNs.

FIG. 13 shows a schematic model for IFNβ-mediated NLRP3 inflammasome inhibition.

### DETAILED DESCRIPTION

Inflammation contributes to the development of autoimmune diseases, including MS. In particular, inflammation triggered by receptors in the innate immune system can have a significant impact on MS and other T-cell mediated autoimmune diseases. IFN-β is used as a first-line treatment for MS patients, yet approximately one-third of MS patients do not respond to IFN-β treatment.

The inventors have found that IFN-β inhibits activity of the NLRP3 inflammasome, a sensor of pathogen- and damage-associated molecular patterns that contributes to innate immunity. The disclosure provides embodiments that demonstrate that IFN-β inhibits NLRP3 inflammasome activity by inducing SOCS1 expression, which in turn inhibits activation of the NLRP3 inflammasome by inhibiting NADPH oxidase.

The disclosure also provides embodiments that demonstrate that IFN-β inhibits mitochondrial ROS generation via an upstream molecule, i.e., Rac1. (See FIG. 13). A non-limiting proposed mechanism of action includes detection of IFN-β by Type-1 IFN receptor (IFNAR), which induces SOCS1, a suppressor of Rac1 activation. SOCS1 downregulates Rac1 by reducing expression of Vav1, a guanine nucleotide exchange factor for Rac1, and by directly destabilizing activated Rac1 (associated with GTP). Downregulation of Rac1 activation decreases ROS generation by mitochondria, and eventually negatively controls activation of the NLRP3 inflammasome. FIG. 13 depicts a non-limiting proposed summary of a mechanism by which IFNβ inhibits activation of the NLRP3 inflammasome.

The disclosure provides embodiments herein that correlate the efficacy of IFN-β therapy in a subject with autoimmune disease to activation of the NLRP3 inflammasome in the subject. In some embodiments, the correlation can suggest that the efficacy of IFN-β therapy is maximized in, or limited to, subjects having an autoimmune disease that depends on NLRP3 inflammasome activity. The correlation, in other embodiments, can suggest that IFN-β therapy is minimally effective or ineffective for treating subjects having an autoimmune disease that is NLRP3-independent. As used herein, “NLRP3-independent autoimmune disease” includes, but not limited to, NLRP3-independent EAE and NLRP3-independent MS. In some embodiments, NLRP3-independent autoimmune disease may be induced by acute microbial infection. In addition, MS that is associated with optic neuritis may be an indication of NLRP3-independent MS, or MS that will not be responsive to therapy comprising IFN-β. Accordingly, the methods described herein relating to identifying a subject or a patient class can further comprise determining that the subject or patient has, or has a history of, acute microbial infection and optic neuritis.

Thus, in a broad sense the disclosure provides methods for assessing the activation state of the NLRP3 inflammasome in a patient with an autoimmune disease such as MS, facilitates predictions regarding the efficacy of a NLRP3 inflammasome inhibitor such as IFN-β, allows identification of non-responders to such drugs, aids selecting drugs for treating patients with autoimmune diseases, and permits optimizing treatment regimens for patients with autoimmune diseases. In addition the methods for screening compounds for NLRP3-inhibitory activity, described herein, can allow for identification of therapies for treating NLRP3-depending autoimmune diseases, other than the widely used IFN-β therapeutic regimen. Conversely, methods that utilize the experimental induction of NLRP3 inflammasome-independent autoimmune disease can allow for identification of new compounds for treating the substantial proportion of MS patients who are clinical “non-responders” to IFN-β treatment.

Throughout this disclosure, various aspects may be presented in a range format. It should be understood that the description in range format is merely for convenience and brevity, and should not be construed as an inflexible limitation on the scope of the disclosure or claims. Accordingly, as will be understood by one skilled in the art, for any and all purposes, particularly in terms of providing a written description, all ranges disclosed herein also encompass any and all possible subranges and combinations of subranges thereof, as well as all integral and fractional numerical values within that range. As only one example, a range of 20% to 40% can be broken down into ranges of 20% to 32.5% and 32.5% to 40%, 20% to 27.5% and 27.5% to 40%, etc. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third, and upper third, etc. Further, as will also be understood by one skilled in the art, all language such as “up to,” “at least,” “greater than,” “less than,” “more than” and the like include the number recited and refer to ranges which can be subsequently broken down into subranges as discussed above. In the same manner, all ratios disclosed herein also include all subratios falling within the broader ratio. These are only examples of what is specifically intended. Further, the phrases “ranging/ranges between” a first indicate number and a second indicate number and “ranging/ranges from” a first indicate number “to” a second indicate number are used herein interchangeably.

Without limiting the disclosure to any particular mechanism of action, it is believed that NLRP3 is regulated at least according to the mechanisms described herein. NLRP3 is a member of the NOD-like receptor (“NLR”) family of intracellular receptors that sense pathogen- and damage-associated molecular patterns. Upon activation, members of the NLR family participate in cytosolic multiprotein complexes known as inflammasomes. The NLR protein NLRP3 can be activated by stimuli such as pore-forming toxins, extracellular ATP, viral DNA, inhaled particulates, uric acid crystals, bacteria, and small-molecule immune activators, among others. For example, extracellular ATP generates a damage signal through the receptor P2X7 that stimulates assembly of the NADPH oxidase complex, which generates reactive oxygen species (“ROS”) critical to NLRP3 inflammasome activation. ROS generation by mitochondria may play more of a role in the activation of the NLRP3 inflammasome (Zhou et al., Nature. 2011 469(7329):221-225). The data described below in the Examples suggests that IFN-β can inhibit mitochondrial ROS generation.

Upon stimulation of the NLRP3 pathway by ATP or another appropriate signal, the guanine nucleotide-binding protein Rac1 transitions from its inactive GDP-bound form to its active GTP-bound form through the activity of its guanine nucleotide exchange factor Vav1. GTP-Rac1 associates with proteins including Nox2 and p47^{phox} to form the active NADPH oxidase complex, enabling production of ROS and contributing to the activation of intracellular NLRP3 protein. Activated NLRP3 oligomerizes and associates with the adaptor protein apoptosis-associated speck-like protein (“ASC”) to form the active NLRP3 inflammasome, which in turn recruits and activates caspase-1 protein. Activated caspase-1 mediates the proteolytic maturation of the cytokines IL-1β and IL-18 into their active secreted forms.

The ability of IFN-β to inhibit NLRP3 activation (and thus IL-1β and IL-18 production) is mediated by a protein known as suppressor of cytokine signaling-1 (“SOCS-1”), which acts by negatively regulating NADPH oxidase activity and/or Rac1 activation and the resulting mitochondrial ROS generation. IFN-β initiates or regulates numerous signaling pathways by binding to its cell-surface receptor IFNAR, and one effect of IFN-β signaling through IFNAR is to upregulate expression of SOCS-1. SOCS-1 downregulates expression of Vav1, which generates GTP-Rac1. Vav1 is directly related to activate Rac1 (GTP-Rac1 is an active form of Rac1, destabilizes active GTP-Rac1, and inhibits p47^{phox} localization within NADPH oxidase complexes or ROS generation by mitochondria. These functions of IFN-β signaling interfere with NADPH oxidase-mediated ROS production and, as a result, with NLRP3 activation. Therefore, IFN-β reduces NLRP3-dependent activation of caspase-1 and production of IL-1β and IL-18.

Experimental autoimmune encephalomyelitis (“EAE”) is an experimentally-induced autoimmune disease of laboratory animal subjects such as mice, rats, guinea pigs, marmosets, rabbits, and non-human primates that has served for decades in basic and translational research animal model for MS. EAE is the most well established and widely used animal model for MS, and the evaluation of therapeutic compounds in EAE models has led to the successful development and introduction of several FDA-approved agents into clinical practice for MS treatment, including glatiramer acetate (Copaxone®) and natalizumab (Tysabri®). EAE may be induced by, for example, administration, through any suitable route, of a composition comprising effective amounts of a suitable immunogen and a suitable adjuvant, optionally followed by or concurrent with additional immunostimulation procedures. For example, EAE may be induced in a murine (mouse) subject by subcutaneously injecting a composition comprising an appropriate amount of a peptide derived from myelin oligodendrocyte glycoprotein (“MOG”), MOG₃₅₋₅₅ peptide (e.g., 100 μg per mouse), emulsified with CFA (e.g., 100 μl per mouse) and heat-killed mycobacteria (e.g., 200, 300, or 400 μg), followed by intraperitoneal (i.p.) injections of Pertussis toxin (e.g., 200 ng per mouse) on the same day and again two days post-injection. One of skill in the art will recognize that other suitable peptides, adjuvants, amounts of peptides and adjuvants, and induction protocols may be used to induce EAE. The symptoms of EAE resemble MS, an autoimmune disease in humans. EAE is characterized by symptoms such as, for example, demyelination and leukocyte infiltration in the CNS, as well as a clinical presentation that may include relapse-remission intervals and may comprise tail weakness, tail paralysis, limb weakness, limb paralysis, sensory loss, optic neuritis, ataxia, muscle weakness, and/or muscle spasms. Recovery from symptoms can be complete or partial and the clinical course can vary in terms of symptoms and severity.

EAE induced by a protocol such as is described above may be characterized as NLRP3-dependent or NLRP3-independent. Induction of EAE in wild-type (WT) mice as described above results in NLRP3-dependent EAE, wherein the mice exhibit increased expression levels of markers for NLRP3 activation such as activated caspase-1 in immune cells and of IL-1β and IL-18 in serum. In addition, these mice demonstrate clinical improvement and decreased expression of markers of NLRP3 activation upon treatment with IFN-β. On the other hand, mice deficient in NLRP3 inflammasome function, such as genetically modified knock-out mice (Nlrp3^{-/-} or Asc^{-/-}), may develop NLRP3-independent EAE disease. These mice lack NLRP3 inflammasome function but nonetheless develop clinically apparent EAE after suitable induction. In contrast to animals with NLRP3-dependent EAE, mice with NLRP3-independent EAE do not demonstrate increased expression levels of markers for NLRP3 activation. Furthermore, animals with NLRP3-independent EAE show no response to IFN-β treatment, similar to the substantial proportion of MS patients that present as non-responders to IFN-β treatment.

As shown in the Examples, NLRP3-inflammasome independent EAE can be induced with aggressive EAE induction regimens in WT mice that have intact NLRP3 inflammasome, which suggests that “aggressive EAE induction regimens” may be mimicked by acute infections in nature. EAE that are induced by aggressive regimens also do not demonstrate expression levels of markers for NLRP3 activation and also do not respond to IFN-β therapy at all.

In broad aspects, the disclosure provides methods related to treatment of an autoimmune disease in a patient. In an aspect, the disclosure provides methods for optimizing a treatment regimen for an autoimmune disease in a patient, where the method comprises obtaining a sample from the patient, determining an expression level of a marker in the sample, and adjusting the treatment regimen for the autoimmune disease in the patient to include or exclude a drug, wherein the treatment regimen includes administration of the drug when the expression level of the marker in the sample is increased relative to a control expression level and wherein the treatment regimen omits administration of the drug when the expression level of the marker in the sample is not increased relative to a control expression level.

In another aspect, the disclosure provides methods for selecting a drug for treating an autoimmune disease in a patient comprising obtaining a sample from the patient, determining an expression level of a marker in the sample, and selecting the drug for treating the autoimmune disease in the patient when the expression level of the marker in the sample is increased relative to a control expression level.

In another aspect, the disclosure provides methods for predicting efficacy of a drug for treating an autoimmune disease in a patient comprising obtaining a sample from a patient and determining an expression level of a marker in the sample, wherein the method predicts high efficacy of the drug when the expression level of the marker in the sample is increased relative to a control expression level and wherein the method predicts low efficacy of the drug when the expression level of the marker in the sample is not increased relative to a control expression level. Efficacy as used in the disclosure should be interpreted as it would be understood by one of skill in the art, and may comprise, but is not limited to, halting or slowing disease progression, reversing the disease, ameliorating disease and/or disease symptoms, minimizing or avoiding deleterious side effects, preventing onset of a disease in a subject that is identified as likely to develop the disease, and the like, where the presence, symptoms, and progression of disease can be observed and/or measured by any suitable method known in the art such as, for example, commonly used clinical evaluation. Furthermore, optimizing a treatment regimen, as used in the disclosure, should be interpreted as designing, adapting, or tailoring a treatment regimen to improve or maximize the efficacy of the treatment regimen.

In another aspect, the disclosure provides methods for identifying a patient with an autoimmune disease as non-responsive to treatment with a drug comprising obtaining a sample from the patient and determining an expression level of a marker in the sample, wherein the patient is identified as non-responsive when the expression level of the marker in the sample is not increased relative to a control expression level.

The various aspects of the disclosure encompass several common embodiments. In some embodiments, the sample may comprise any suitable material derived from any cell, tissue, organ, fluid, or excretion. Non-limiting examples include, but are not limited to, blood, cerebrospinal fluid, serum, buffy coat, lymphocytes, leukocytes, monocytes, macrophages, splenocytes, plasma, stool, saliva, nasal fluid, urine, ascites, vitreous, biopsy material, mucosal fluid, and the like. The sample may be obtained through any appropriate clinical or laboratory procedure known in the art, including but not limited to, venipuncture, lumbar puncture, biopsy, finger prick, orbital sinus collection, and the like. Furthermore, in some embodiments the sample may be processed to substantially purify a cell type in the sample. The cell type may comprise, but is not limited to, lymphocytes, B-cells, T-cells, leukocytes, monocytes, macrophages, granulocytes, glial cells, microglia, neutrophils, eosinophils, dendritic cells, macrophages, NK cells, basophils, and/or mast cells. As used in the disclosure, substantially purify may mean enriching the cell type in the sample to comprise at least about 50%, at least about 60%, at least about 70%, at least about 80%, at least about 90%, or at least about 95% of the cells remaining in the sample after processing the sample to substantially purify the cell type.

In some embodiments, the marker may be any biomarker (e.g., genetic or biochemical marker) that can correlate to NLRP3 activity such as, for example, activated caspase-1 (including the p10 and/or p20 subunits of activated caspase-1), IL-1β, IL-18, p47^{phox} foci, caspase-1 foci, GTP-Rac1, ASC, NLRP3, or ROS. The expression level of the marker may be determined by any suitable method known to one of skill in the art. In some embodiments, the expression level of the marker may be determined by known methods including, but not limited to, ELISA, ELISPOT, HPLC, confocal microscopy, immunohistochemistry, immunofluorescence, flow cytometry, DHE oxidation, western blot, northern blot, RT-PCR quantitative PCR, quantitative RT-PCR, FACS, immunoprecipitation, RIA, in situ hybridization, microarray hybridization, and the like.

In some embodiments, a control expression level of the marker may derive from a source characterized as having a relatively low, minimal, or baseline expression level of the marker as may be associated with, for example, a relatively low level of NLRP3 inflammasome activation, a minimal level of NLRP3 inflammasome activation, relatively inactive NLRP3 inflammasome, and/or a baseline level of NLRP3 inflammasome activation. Such a control expression level may comprise, for example, an expression level of the marker in a patient or subject not having an autoimmune disease, an expression level of the marker in the same patient or subject before the onset of an autoimmune disease, an expression level of the marker in a patient having NRLP3-independent autoimmune disease, an expression level of the marker in a patient or subject having an autoimmune disease that is a non-responder to IFN-β therapy, an average expression level of the marker in an appropriate population of patients or subjects, or any other appropriate source for a control expression level that would be apparent to one of skill in the art. Some embodiments provide a control expression level taken from a database that includes gene and/or protein expression level data. In embodiments where the control expression level of the marker derives from a source characterized has having a relatively low, minimal, or baseline expression level of the marker, an increased expression level of the marker in a sample relative to the control expression level of the marker may indicate NLRP3-dependent autoimmune disease and/or responsiveness to IFN-β treatment in the patient or subject from which the sample was obtained. In some embodiments, an increased expression level of a marker relative to a control expression level may comprise an expression level at least about 1.1-fold higher, 1.2-fold higher, 1.3-fold higher, 1.4-fold higher, 1.5-fold higher, at least about 2-fold higher, at least about 3-fold higher, at least about 5-fold higher, or at least about 10-fold or higher than a control expression level of the marker.

In some embodiments, a control expression level of the marker may derive from a source characterized as having a relatively high or elevated expression level of the marker as may be associated with, for example, a relatively high level of NLRP3 inflammasome activation, a relatively elevated level of NLRP3 inflammasome activation, and/or relatively active NLRP3 inflammasome. Such a control expression level may comprise, for example, an expression level of the marker in a patient or subject having an NRLP3-dependent autoimmune disease, an expression level of the marker in a patient or subject having an autoimmune disease that is a responder to IFN-β therapy, an average expression level of the marker in an appropriate population of patients or subjects, or any other appropriate source for a control expression level that would be apparent to one of skill in the art. Some embodiments provide a control expression level taken from a database that includes gene and/or protein expression level data. In embodiments where the control expression level of the marker derives from a source characterized has having a relatively high or elevated expression level of the marker, a decreased expression level of the marker in a sample relative to the control expression level of the marker may indicate NLRP3-independent autoimmune disease and/or non-responsiveness to IFN-β treatment in the patient or subject from which the sample was obtained. In some embodiments, a decreased expression level of a marker relative to a control expression level may comprise an expression level at least about 1.1-fold lower, 1.2-fold lower, 1.3-fold lower, 1.4-fold lower, 1.5-fold lower, at least about 2-fold lower, at least about 3-fold lower, at least about 5-fold lower, or at least about 10-fold or lower than the control expression level of the marker.

In some embodiments, the drug comprises an inhibitor of NLRP3 inflammasome activity. In some embodiments, the inhibitor of NLRP3 inflammasome activity may comprise IFN-β, including, but not limited to, IFN-β, any fragments, fusions, or modified versions thereof that retain the ability to inhibit NLRP3 inflammasome activity, any commercial preparations comprising IFN-β, and/or any drugs comprising IFN-β and approved for MS treatment such as IFN-β-1a produced in cultured mammalian cells (Rebif® or Avonex®) and/or IFN-β-1b produced in E. coli (Betaseron° or Betaferon®). In some embodiments, a treatment regimen for an autoimmune disease may comprise drugs such as, but not limited to, IFN-β and/or another inhibitor of NLRP3 inflammasome activity, glatiramer acetate, natalizumab, mitoxantrone, laquinimod, alemtuzumab, rituximab, teriflunomide, fingolimod, daclizumab, BG00012, cladribine, and/or a corticosteroid. Some embodiments provide for combination therapy comprising any combination of two or more inhibitors of NLRP3 inflammasome activity.

In another aspect, the disclosure provides methods for identifying a compound capable of inhibiting NLRP3 inflammasome activity comprising contacting a cell with a test compound, contacting the cell with an activator of NLRP3 inflammasome activity, and determining whether the test compound inhibits NLRP3 inflammasome activity in the cell. In some embodiments, the cell may be a cell such as, but not limited to, a macrophage, monocyte, splenocyte, lymphocyte, leukocyte, B-cell, T-cell, NK-cell, granulocyte, glial cell, microglial cell, neutrophil, eosinophil, mast cell, basophil, dendritic cell, neural cell, fibroblast, kidney cell, or epithelial cell. And in some embodiments, the cell may be comprise and/or derive from a cell or cell source such as, but not limited to primary cell, a tissue explant, a blood sample, an immortalized cell line, a genetically modified cell, a transformed cell, a transduced cell, a transfected cell, and the like. The cell may be maintained by any appropriate method known to one of skill in the art.

In some embodiments, the activator of NLRP3 inflammasome activity may comprise one or more activators of NLRP3 inflammasome activity such as, but not limited to, ATP, viral DNA, a virus, a fungal organism, a bacteria, silica, asbestos, a skin irritant, UV light, amyloid β protein, calcium pyrophosphate dehydrate, hyaluronan, and/or alum.

In some embodiments, the determining step may comprise measuring expression of a marker of NLRP3 inflammasome activity, such as, but not limited to activated caspase-1 (including p10 and/or p20 subunits of activated caspase-1), IL-1β, IL-18, p47^{phox} foci, caspase-1 foci, GTP-Rac1, ASC, NLRP3, or ROS. The expression level of the marker may be determined by any suitable method known to one of skill in the art. In some embodiments, the expression level of the marker may be determined by known methods including, but not limited to, ELISA, ELISPOT, HPLC, confocal microscopy, immunohistochemistry, immunofluorescence, flow cytometry, DHE oxidation, western blot, northern blot, RT-PCR quantitative PCR, quantitative RT-PCR, FACS, immunoprecipitation, RIA, in situ hybridization, microarray hybridization, and the like.

In some embodiments, the determining step may comprise reviewing the medical history of the patient for a recent history of any type of microbial infections and/or optic neuritis in combination with measuring expression of a marker of NLRP3 inflammasome activity. In some embodiments, a disease history of patients with optic neuritis may be an indication of a possible IFNβ non-responder. A history of severe microbial infection may be indicative that a strong innate inflammation was triggered and caused the development of EAE (i.e., the non-human version of MS) in non-human animals without activating the NLRP3 inflammasome. In some embodiments, a disease history of patients with microbial infections, in addition to the absence of NLRP3 inflammasome activity, may be an indication of a possible IFNβ non-responder (i.e., NLRP3-independent autoimmune disease). Examples of microbial infections include but not limited to post operational infections, bacterial pneumonia infections, sepsis, skin infections, wound infection, osteomyelitis, skin polymicrobial infections, allergies, asthma, endocarditis, arthritis, abscess, sinusitis, and acne vulgari. Such infections may be caused by viruses, bacteria, fungi, algae and protozoa. In some embodiments the acute infection comprises a bacterial infection.

In some embodiments, the determining step may comprise measuring T cell infiltration in the brain and/or spinal cord. In some embodiments, a high ratio of T cell infiltration in the brain compared to the spinal cord may be an indication of a possible IFNβ non-responder (i.e., NLRP3-independent disease). In some embodiments, the number of infiltrated T cells can be measured indirectly (e.g., by assessing the subject for other indications of T cell infiltration, or likelihood of T cell infiltration). The T cell infiltration may be measured by any appropriate method known to one of skill in the art.

In another aspect, the disclosure provides methods for identifying a therapeutic compound for treating an NLRP3-independent autoimmune disease in a subject comprising administering a test compound to the subject and determining whether the autoimmune disease in the subject responds to the test compound. In some embodiments, the NLRP3-independent autoimmune disease may be MS, EAE, or another suitable model for a human autoimmune disease that is independent of NLRP3 inflammasome activity. In some embodiments, the subject may be a mammal, such as, but not limited to, a mouse, a human, a rat, a guinea pig, a marmoset, a rabbit, or a non-human primate. In some embodiments, the subject may comprise a genetic mutation and/or modification, for example, the subject may be deficient in expression of one or more genes or gene products such as, but not limited to, NLRP3, ASC, IFNAR, caspase-1 and/or IFN-β.

In some embodiments, the disease may be considered to respond to the test compound where, for example, administration of the test compound results in halting or slowing disease progression, reversing disease, ameliorating disease symptoms, preventing onset of a disease in a subject that is identified as likely to develop the disease, and the like, where the presence, symptoms, and progression of disease can be observed and/or measured by any suitable method known in the art.

In another aspect, the disclosure provides methods of treating an NLRP3-independent autoimmune disease and/or IFNβ-non-responsive MS in a subject. In some embodiments of this aspect, the method comprises administering to the subject an effective amount of an agent that inhibits the chemokine receptor, CCR2. In embodiments of this aspect, the agent that inhibits CCR2 can be any compound known in the art that can inhibit CCR2 function. In some embodiments the agent can inhibit a CCR2 from any mammal such as, for example, mouse (e.g., GenBank Accessions NM_{—}009915 and NP_{—}034045.1) or human (e.g., GenBank Accessions NG_{—}021428, NM_{—}001123396, NM_{—}001123041, NP_{—}001116868.1, and NP_{—}001116513.2), inclusive of variants and mutants thereof In some embodiments, the agent can be a specific inhibitor (e.g., small molecule, antibody, siRNA, miRNA, etc.) or a non-specific inhibitor of CCR2 (e.g., binding to ligands of CCR2). Examples of inhibitors of CCR2 are described in Doyon et al., Chem Med Chem. 3(4):660-669 (2008), U.S. Patent Publication No. 20100234364 and U.S. Patent Publication No. 20110144129, each incorporated by reference herein in their entirety. In some embodiments, the CCR2 inhibitor may comprise INCB3344 (Pfizer). In some embodiments of the method, the subject may be a mammal such as, for example a mouse or a human. In embodiments wherein the subject is a human, the NLRP3 inflammasome-independent autoimmune disease may be multiple sclerosis.

In certain embodiments, the CCR2 inhibitor is administered in a pharmaceutically acceptable composition, such as in or with a pharmaceutically acceptable carrier. In such embodiments, pharmaceutical compositions can be formulated in a conventional manner using one or more physiologically acceptable carriers or excipients.

“Pharmaceutically acceptable” means suitable for use in a human or other mammal. The terms “pharmaceutically acceptable carriers” and “pharmaceutically acceptable excipients” are used interchangeably and refer to substances that are useful for the preparation of a pharmaceutically acceptable composition. In certain embodiments, pharmaceutically acceptable carriers are generally compatible with the other ingredients of the composition, not deleterious to the recipient, and/or neither biologically nor otherwise undesirable.

Certain exemplary pharmaceutically acceptable carriers include, but are not limited to, substances useful for topical, ocular, parenteral, intravenous, intraperitoneal intramuscular, sublingual, nasal and oral administration. “Pharmaceutically acceptable carrier” also includes agents for preparation of aqueous dispersions and sterile powders for injection or dispersions. Examples of pharmaceutically acceptable carriers and excipients are discussed, e.g., in Remington Pharmaceutical Science, 16th Ed. Certain exemplary techniques and compositions for making dosage forms are described in the following references: Modern Pharmaceutics, Chapters 9 and 10, Banker & Rhodes, eds. (1979); Lieberman et al., Pharmaceutical Dosage Forms: Tablets (1981); and Ansel, Introduction to Pharmaceutical Dosage Forms, 2nd Ed., (1976).

“Administering” refers to administration of agents as needed to achieve a desired effect. Exemplary routes of administration include, but are not limited to, oral, rectal, nasal, sublingual, buccal, intramuscular, subcutaneous, intravenous, transdermal, and parenteral administration. Such administration can be, in certain embodiments, by injection, inhalation, or implant.

It is desirable that the route of administration and dosage form of the preparation be selected to maximize the effect of the treatment. Typical examples of the administration route include oral routes as well as parenteral routes, including intracerebral, intraperitoneal, intraoral, intrabronchial, intrarectal, subcutaneous, intramuscular and intravenous routes. Typical examples of the dosage form include sprays, capsules, liposomes, tablets, granules, syrups, emulsions, suppositories, injections, ointments and tapes.

One skilled in the art can select an appropriate dosage and route of administration depending on the patient, the particular autoimmune disease being treated, the duration of the treatment, concurrent therapies, etc. In certain embodiments, a dosage is selected that balances the effectiveness with the potential side effects, considering the severity of the autoimmune disease.

For oral therapeutic administration, the composition may be combined with one or more carriers and used in the form of ingestible tablets, buccal tablets, troches, capsules, elixirs, suspensions, syrups, wafers, chewing gums, foods and the like. Such compositions and preparations should contain at least 0.1% of active compound. The percentage of the compositions and preparations may, of course, be varied and may conveniently be between about 0.1 to about 100% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring. The above listing is merely representative and one skilled in the art could envision other binders, excipients, sweetening agents and the like. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like.

The selected dosage level will depend upon a variety of factors including the activity of the particular compound of the present invention employed, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

In general, the daily dose contains from about 0.1 mg to about 2000 mg. More preferably, each dose of a compound contains about 0.5 to about 60 mg of the active ingredient. This dosage form permits the full daily dosage to be administered in one or two oral doses. More than once daily or twice daily administrations, e.g., 3, 4, 5 or 6 administrations per day, are also contemplated herein.

As used herein, an “amount effective” (or “effective amount”) refers to a sufficient amount of an agent or a compound being administered which will relieve to some extent one or more of the symptoms of the disease or condition being treated. The result can be reduction and/or alleviation of the signs, clinical indications or symptoms, or causes of a disease, or any other desired alteration of a biological system. Accordingly, methods of treatment as disclosed herein can slow or halt the progression of a disease, or reverse a disease, such as an autoimmune disease. An appropriate “effective” amount in any individual case may be determined using techniques, such as a dose escalation study.

In another aspect, the disclosure provides a kit for performing any of the methods disclosed herein, wherein the kit includes a buffer and at least one member of a specific binding pair (e.g., oligonucleotide for amplifying or binding to a marker (e.g., DNA, mRNA, etc.) or an antibody, etc.) that can bind to a marker and comprising a detectable label, allowing for the detection and quantification of the marker in a biological sample from a subject. In some embodiments, the marker may be any biomarker (e.g., genetic (e.g., nucleic acid) or biochemical (e.g., carbohydrate, protein, etc.) marker) that can correlate to NLRP3 activity such as, for example, activated caspase-1 (including the p10 and/or p20 subunits of activated caspase-1), IL-1β, IL-18, p47^{phox} foci, caspase-1 foci, GTP-Rac1, ASC, NLRP3, or ROS. In some embodiments, the oligonucleotide primer may be used to amplify IL-1β or IL-18. In some embodiments, the antibody specifically binds to IL-1β or IL-18. The kit can incorporate a detectable label as known in the art such as, for example, a fluorophore, radioactive moiety, enzyme, biotin/avidin label, chromophore, chemiluminescent label, or the like. The kit can include reagents for labeling the oligonucleotide and/or antibodies or include reagents for detecting oligonucleotide (e.g., labeled microparticles and/or labeled complementary nucleotides, a microarray, etc.) and/or the antibodies (e.g., detection antibodies) and/or for labeling the markers (if present) or reagents for detecting the markers (if present). In some embodiments, the kit may also optionally include reagents required to perform any of the methods disclosed herein, such as buffers, salts, enzymes, enzyme co-factors, substrates, detection reagents, and the like. The kit may additionally include one or more other controls or reference (baseline) values for one or more markers. The kits may also include vials, containers, and other packaging materials for storing the above reagents. Instructions included in kits can be affixed to packaging material or can be included as a package insert. While the instructions are typically written or printed materials they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

All references disclosed herein, including but not limited to patents, patent applications, and non-patent literature are hereby incorporated by reference in their entireties for all purposes.

While various aspects and embodiments have been disclosed herein, other aspects and embodiments will be apparent to those skilled in the art. The various aspects and embodiments disclosed herein are merely for purposes of illustration and clarity, and are not intended to be limiting to the scope of the appended claims. Similarly, the Examples that follow are merely illustrative of certain aspects and embodiments and are not to be taken as limiting in any way.

### EXAMPLES

### Example 1

### IFN-β Signal Suppresses IL-1β via NLRP3

We examined the role of interferon-β (IFN-β) signaling on aspects of inflammation response through the experimental evaluation of wild-type and IFNAR-deficient mice. Our experiments, detailed below, demonstrate that IFN-β signaling suppresses IL-1β production through inhibition of NLRP3 inflammasome. Suppression of NLRP3 inflammasome activity by IFN-β was demonstrated by production of IL-1β, activation of caspase-1, and formation of intracellular foci comprising oligomers of activated caspase-1.

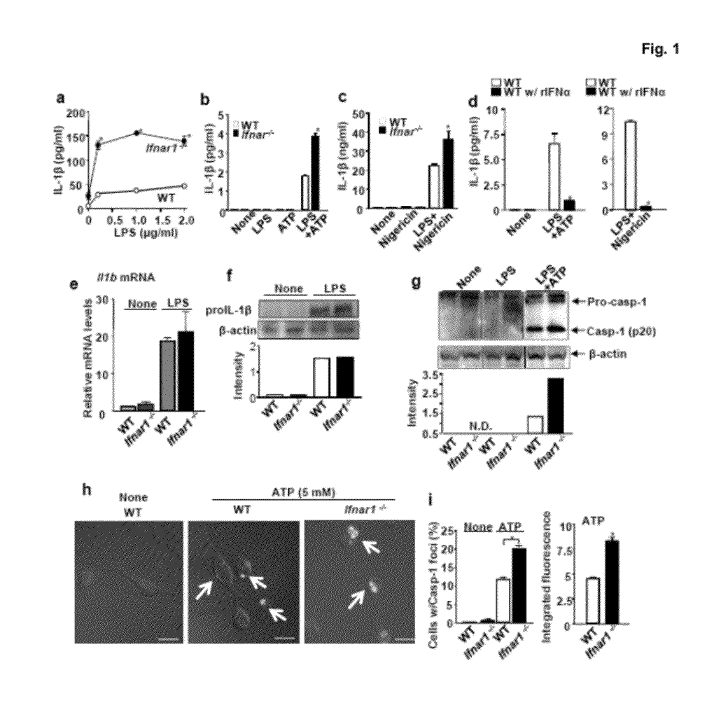

Wild-type and Ifnar1^{-/-} dendritic cells were incubated with 0, 0.2, 1.0, or 2.0 μg/ml LPS for 48 hours, and the concentration of secreted IL-1β was measured by ELISA. See FIG. 1(a). Peritoneal macrophages isolated from wild-type and Ifnar1^{-/-} mice were incubated with Ultrapure LPS alone (100 ng/ml), ATP alone (5 mM), or a combination of Ultrapure LPS and ATP (100 ng/ml LPS+5 mM ATP) for 24 hours, and the concentration of secreted IL-1β was measured by ELISA. See FIG. 1(b). In addition, peritoneal macrophages isolated from wild-type and Ifnar1^{-/-} mice were incubated with nigericin alone (2.5 μM) or combination of Ultrapure LPS (100 ng/ml) and nigericin (2.5 μM) for 24 hours. See FIG. 1(c). In each case, the cells lacking IFNAR expression responded to NLRP3 induction with greater secretion of IL-1β, indicating more pronounced NLRP3 inflammasome activity than in wild-type cells with an intact IFN-β signaling pathway.

Bone marrow-derived macrophages were isolated from wild-type mice and incubated for 24 hours with or without 1,000 units/ml recombinant IFN-α (rIFNα, another activator of IFNAR signaling). After a 24 hour incubation, the cells were exposed to Ultrapure LPS (100 ng/ml) and either ATP (5 mM) or nigericin (2.5 μM) for 4 h (with the ATP or nigericin added for the last hour). Secretion of IL-1β was then measured by ELISA. Induction of IFNAR signaling before exposure to NLRP3 inflammasome activators reduced IL-1β production, see FIG. 1(d). The presence of IFNAR, however, does not appear to affect the expression of the IL-1β precursor pro-IL-1β. Peritoneal macrophages isolated from wild-type or Ifnar1^{-/-} mice were incubated with or without 100 ng/ml Ultrapure LPS for 3 hours. The cells from each sample were then harvested and divided into two samples. Total RNA was purified from one set of cell samples using TRIzol reagent (Invitrogen™), and IL-1β mRNA in each sample was measured by quantitative RT-PCR and normalized to the sample representing wild-type cells with no LPS exposure. See FIG. 1(e). Total protein was purified from the other set of cell samples by lysing the cells in SDS-PAGE sample buffer, and proteins in the cell lysates were separated by electrophoresis on a 10% polyacrylamide gel. The proteins were then transferred to a nitrocellulose membrane and probed for the presence of pro-IL-1β by western blot using a rabbit anti-IL-1β antibody (Cell Signaling Technology), followed by an HRP-conjugated donkey anti-rabbit antibody (Pierce) and chemiluminescent detection (ECL reagents, GE Healthcare) to visualize the ˜31 kDa band representing pro-IL-1β. The same membranes were stripped and probed again by western blot to detect β-actin, and pro-IL-1β signal densitometry for each sample was normalized to β-actin using NIH Image/Image J software. See FIG. 1(f).

In addition, IFNAR signaling inhibits NLRP3 inflammasome activity as measured by caspase-1 activation. Peritoneal macrophages isolated from wild-type and Ifnar1^{-/-} mice were incubated with or without 100 ng/ml Ultrapure LPS or 100 ng/ml Ultrapure LPS plus 5 mM ATP for 4 h (ATP was applied for the last hour). Cell lysates were prepared for western blot as described above and probed for caspase-1 expression using a using a rabbit anti-caspase-1 antibody (Sigma Aldrich), followed by an HRP-conjugated donkey anti-rabbit antibody (Pierce) and chemiluminescent detection (ECL reagents, GE Healthcare) to visualize the ˜20 kDa band representing active caspase-1 p20, as well as the ˜45 kDa band representing inactive pro-caspase-1. The same membranes were stripped and probed again by western blot to detect β-actin, and caspase-1 p20 signal densitometry for each sample was normalized to β-actin using NIH Image/Image J software. See FIG. 1(g). Next, peritoneal macrophages isolated from wild-type and Ifnar1^{-/-} mice were incubated with or without 5 mM ATP for 30 min at 37° C. Cells were then fixed, probed for caspase-1 protein (red) with a rabbit anti-caspase-1 antibody (Sigma Aldrich), followed by an AlexaFluor® 594-conjugated goat anti-rabbit antibody (Invitrogen™), stained with DAPI (blue), and subjected to confocal fluorescence microscopy to detect the frequency of caspase-1 foci. See FIGS. 1(h)-(i). Upon the application of NLRP3 activators LPS and/or ATP, caspase-1 activation was more pronounced in Ifnar1^{-/-} cells whether measured by expression of mature caspase-1 p20 or the frequency of caspase-1 foci in cells.

### Example 2

### Mechanism by which IFNAR Signaling Suppresses NLRP3 Activity

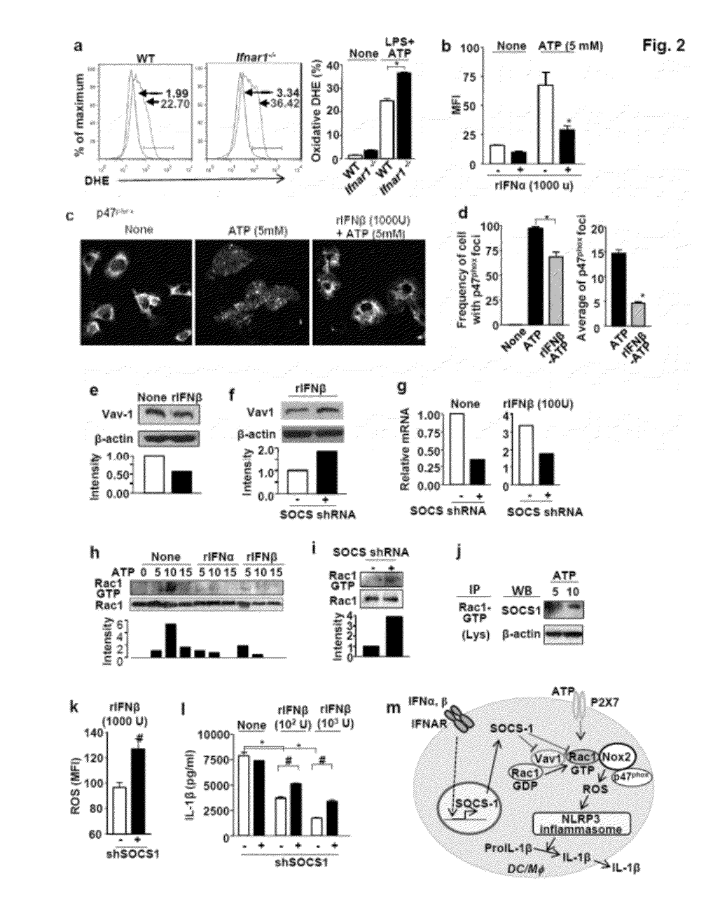

IFNAR signaling suppresses NLRP3 inflammasome activity, and we have delineated the likely mechanism by which IFNAR signaling suppresses NLRP3 inflammasome activity. First, stimulation of IFNAR by IFN-β inhibits ATP-induced ROS production. Bone marrow-derived macrophages obtained from wild-type and Ifnar1^{-/-} mice were incubated with (red lines) or without (black lines) 100 ng/ml Ultrapure LPS and 5 mM ATP for 1 hour (ATP was added in the last 0.5 h), and ROS production was detected by DHE oxidation by flow cytometry. Cell populations deficient in IFNAR signaling exhibited more ROS production. See FIG. 2(a). In addition, IFN pretreatment decreased ROS production upon exposure to ATP. Bone marrow-derived macrophages from wild-type mice were incubated with or without 1,000 units/ml rIFN-α for 24 hours. The cells were then exposed to 5 mM ATP for 1 hour, and ROS production was detected by DHE oxidation and flow cytometry. See FIG. 2(b); MFI represents the mean fluorescence intensity of DHE staining.

IFNAR signaling inhibits ROS production by inhibiting NADPH oxidase activity. Pretreatment of bone marrow-derived macrophages obtained from wild-type mice with 1,000 units/ml rIFN-β inhibited the formation of p47^{phox} foci indicative of activated NADPH oxidase complexes. Cells incubated with and without IFN-β pretreatment for 24 hours were then exposed to 5 mM ATP for 1 hour, fixed, and probed with a FITC-conjugated anti-p47^{phox} antibody to detect foci representing active NADPH oxidase. See FIG. 2(c). Pretreatment with IFN-β caused reductions in both the frequency of cells exhibiting foci and the average number of foci per cell. See FIG. 2(d).

IFN-β interferes with NADPH oxidase signaling by downregulating Vav1 expression. Bone marrow-derived macrophages from wild-type mice were pretreated with 1000 units/ml rIFNα for 24 hours before treatment with 5 mM ATP for one hour. Cell lysates were prepared for western blot as described above and probed for Vav1 expression using a using a rabbit anti-Vav1 antibody (Cell Signaling Technology), followed by an HRP-conjugated donkey anti-rabbit antibody (Pierce) and chemiluminescent detection (ECL reagents, GE Healthcare). The same membranes were stripped and probed again by western blot to detect β-actin, and Vav1 signal densitometry for each sample was normalized to β-actin using NIH Image/Image J software. See FIG. 2(e). IFN-β also decreases cellular levels of GTP-Rac1. Bone marrow-derived macrophages obtained from wild-type mice were pretreated with 1,000 units/ml rIFNα or IFNβ for 24 hours before treatment with 5 mM ATP for 5, 10, or 15 minutes. Cells were then lysed, and total GTP-Rac1 was compared to GTP-Rac1 by western blot first using an antibody specific for GTP-Rac1, followed by membrane stripping and probing with an antibody recognizing total Rac1. GTP-Rac1 signal densitometry for each sample was normalized to total Rac1 using NIH Image/Image J software. See FIG. 2(h).

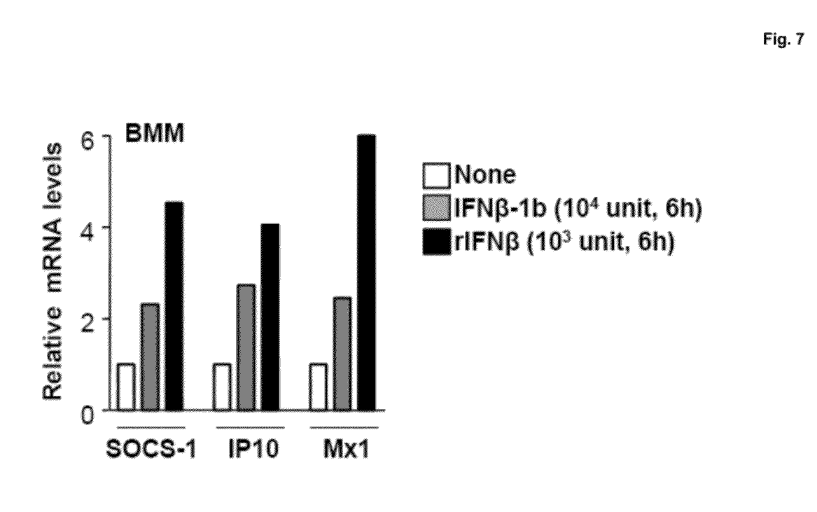

IFN-β mediates its effects on NADPH oxidase activity through SOCS-1. First, IFN-β induces SOCS-1 expression. Bone marrow-derived macrophages derived from wild-type mice were incubated with no treatment, 1,000 units/ml recombinant mouse IFN-β, or 10,000 units/ml human recombinant IFN-β-1b (Betaseron®) for 6 hours. Total RNA was purified from each group of cells using TRIzol® reagent (Invitrogen™), and SOCS-1, IP10, and Mx1 mRNA in each sample was measured by quantitative RT-PCR, and values were normalized to the sample with no IFN-β exposure. See FIG. 7. IP10 and Mx1 are known to be induced IFN-β, and SOCS-1 expression increased to a similar degree as IP10 and Mx1 in the presence of IFN-β. In addition, a decrease in SOCS-1 expression increases cellular levels of Vav1 and GTP-Rac1. Mouse bone marrow-derived macrophages were transduced with a lentiviral expression vector expressing a shRNA that decreases SOCS-1 expression (see FIG. 2(g)). Four hours later, rIFN-β was added (1,000 units/ml), and the cells were incubated for 24 hours. Cells were then lysed and probed for Vav1 expression by western blot as described above, revealing increased Vav1 expression in the anti-SOCS-1 shRNA-transduced cells. See FIG. 2(f). Similarly, the same shRNA treatment also increased GTP-Rac1 levels in transduced cells, see FIG. 2(i). SOCS-1 also mediates inhibition of ROS and IL-1β production by IFN-β. Bone marrow-derived macrophages were treated with 100 ng/ml Ultrapure LPS and 5 mM ATP, with or without lentiviral transduction with the anti-SOCS-1 shRNA. ROS was detected by flow cytometry as described, see FIG. 2(k), and IL-1β secretion was detected by ELISA using standard techniques, see FIG. 2(l). In addition, SOCS-1 associates with Rac1-GTP as detected by immunoprecipitation. Lysates from bone marrow-derived macrophages were treated with 5 mM ATP for 5 or 10 minutes and then immunoprecipitated with either anti-GTP-Rac1, followed by anti-SOCS-1 western blot, all using standard techniques. See FIG. 2(j).

These data lead to a model in which IFN-β inhibits assembly of the NADPH oxidase complex, resulting in reduced generation of ROS, an NLRP3 inflammasome activator. See FIG. 2(m). IFN-β inhibits ROS generation and activity of NADPH oxidase, which is a direct upstream event of ROS generation. Suppression of NADPH oxidase is achieved by negative regulation of Vav1 expression (which is essential for Rac1 activation), de-stabilization of active Rac1, and p47phox intracellular translocation to form the complete NADPH oxidase molecular complex. These events are mediated by SOCS1 in an IFNAR signaling-dependent fashion.

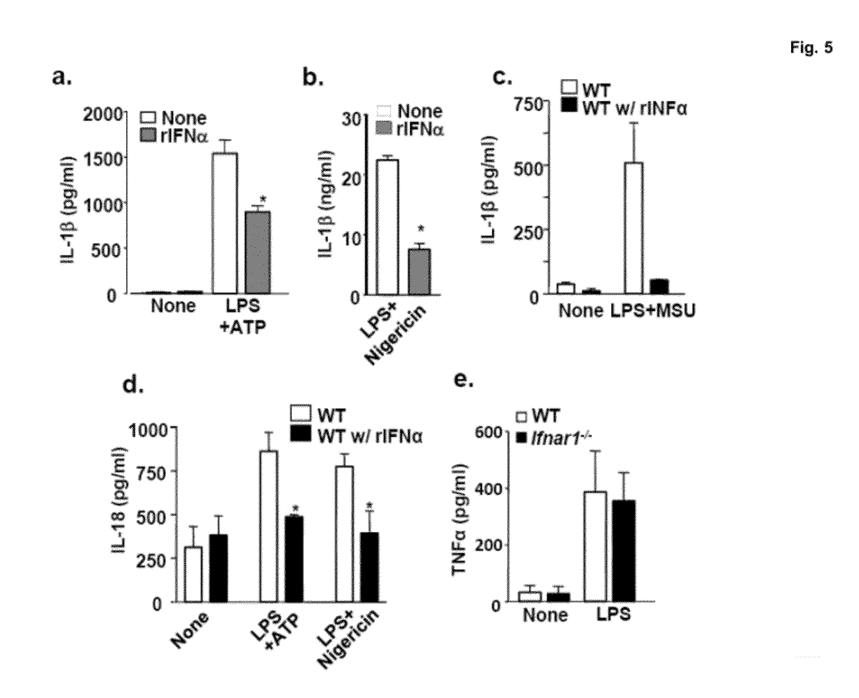

IFNAR signaling suppresses NLRP3 inflammasome activity as shown in FIG. 5. Peritoneal macrophages (FIG. 5(a), (b)) or bone marrow-derived macrophages from wild-type mice were incubated with 100 ng/ml Ultrapure LPS plus 5 mM ATP (a), 2.5 μM nigericin (b), or MSU (c) for 3 hours in the presence or absence of 1,000 units/ml rIFNα. rIFNα was added 24 hours before starting the Ultrapure LPS treatment. Secreted IL-1β concentrations were measured in the cell culture media by ELISA using standard techniques. Similarly, IL-18 protein expression was decreased in culture supernatant of bone marrow-derived macrophages by IFN. Cells were incubated with 100 ng/ml Ultrapure LPS and 5 mM ATP or 2.5 μM nigericin for 24 hours in the presence or absence of 1,000 units/ml rIFNα, and IL-18 secretion was measured in the supernatant by ELISA using standard techniques. See FIG. 5(d). Peritoneal macrophages from wild-type or Ifnar1^{-/-} mice were stimulated with or without 100 ng/ml Ultrapure LPS for 24 h, and expression of TNFα were analyzed by ELISA using the culture supernatants. See FIG. 5(e).

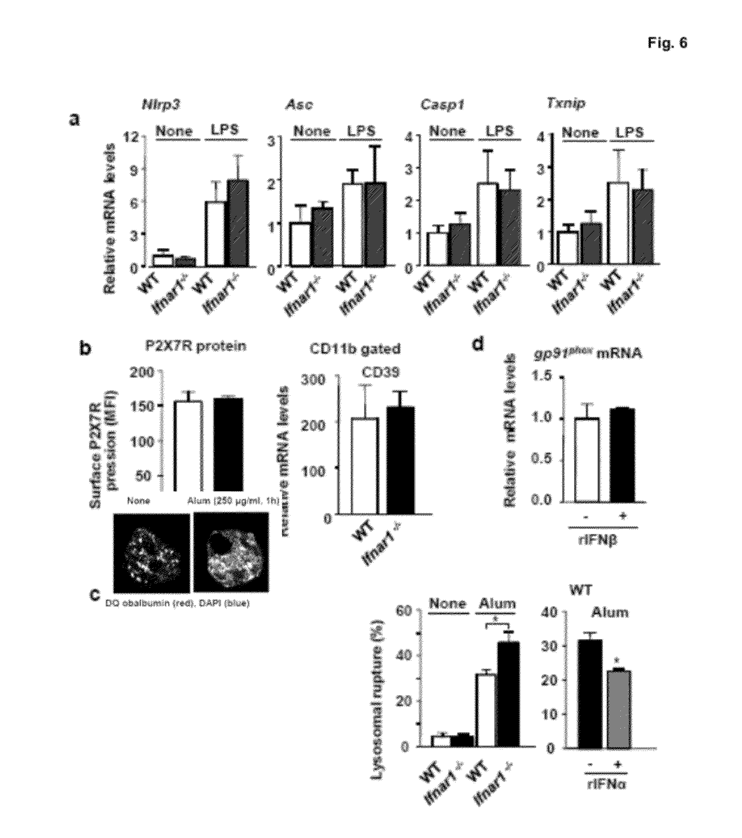

IFNAR signaling does not affect expression of NLRP3 inflammasome components, P2X7R, and CD39, but does induce lysosomal stabilization. See FIG. 6. Nlrp3, Asc, Casp1 and Txnip mRNA expression were evaluated by qRT-PCR. Bone marrow-derived macrophages from wild-type and Ifnar1^{-/-} mice were incubated with or without 100 ng/ml Ultrapure LPS for 3 hours, and the mRNA expression for each gene was measured by RT-PCR using standard techniques, showing no significant differences between the samples. See FIG. 6(a). Similarly, cell surface expression of ATP receptor P2X7R and CD39 (nucleoside triphosphate diphosphohydrolase 1), an ectonucleotidase that hydrolyzes extracellular ATP, on wild-type and Ifnar1^{-/-} macrophages were determined to be equivalent by flow cytometry using standard techniques. See FIG. 6(b). IFN-β exposure also did not affect gp91^{phox} mRNA expression. Wild-type bone marrow-derived macrophages were incubated with or without 1,000 units/ml rIFNβ for 24 hours before determining levels of gp91^{phox} mRNA in the total RNA by qRT-PCR using standard techniques. See FIG. 6(c). IFNAR signaling suppresses Alum-induced lysosomal rupture as demonstrated by confocal microscopy images of DQ ovalbumin (red) and DAPI (blue) staining (left) obtained using standard techniques. DQ ovalbumin staining locates lysosomes. See FIG. 6(e). The proportion of cells with DQ ovalbumin leakage to cytoplasm due to ruptured lysosomes was evaluated by incubating wild-type and Ifnar1^{-/-} macrophages with DQ ovalbumin (10 μg/ml) alone or a combination of DQ ovalbumin (10 μg/ml) and Alum (250 μg/ml) for 1 hour. Wild-type cells were also measured with and without exposure to rIFNα. See FIG. 6(f).

### Example 3

### IFN-β Signaling Suppresses NLRP3 and EAE in Vivo

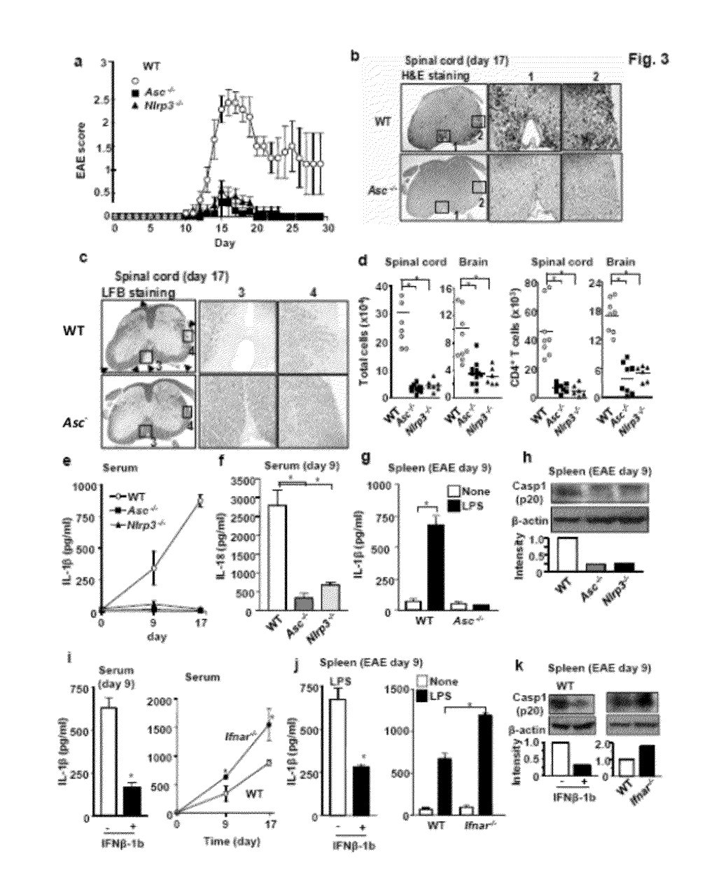

The absence of NLRP3 inflammasome components NLRP3 and ASC ameliorates EAE progression by reducing inflammatory cell infiltration to the CNS (see FIGS. 3(b), (d)), resulting in decreased demyelination (FIG. 3(c)). Levels of NLRP3 inflammasome activation can be assessed by checking IL-1β levels in serum, and levels of IL-1β production and caspase-1 activation by activated splenocytes (FIGS. 3(e)-(h)). We also confirmed that IFN-β treatment suppressed NLRP3 inflammasome activity in vivo by monitoring IL-1β and activated caspase-1 levels (FIG. 3(i)-(j)). These data suggested that NLRP3 inflammasome activation is measurable from samples obtained from mice with EAE.

EAE was induced in Asc−/−, Nlrp3−/−, and wild-type mice. For EAE induction, MOG35-55 peptide (100 μg/mouse) was emulsified with CFA (100 μl/mouse, including 200 μg of heat-killed Mycobacteria), and subcutaneously injected in the flanks of mice on day 0. Mice were also i.p. injected with Pertussis toxin (200 ng/200 μl PBS/mouse) on days 0 and 2. Representative data from one of three independent experiments with disease scores presented as mean±SEM for each group (n=5). (FIGS. 3(b), (c)). Standard hematoxylin and eosin staining (FIG. 3(b)) and LFB staining (FIG. 3(c)) of spinal cord sections from wild-type and Asc−/− mice 17 days after EAE induction demonstrate decreased pathology in Asc−/− mice. Numbered squares indicate representative regions of cell infiltration (H&E) and demyelination (LFB) shown at high magnification. Arrowheads indicate regions of demyelination. The NLRP3-inflammasome is activated during EAE progression. Serum IL-1β (FIG. 3(e)) and IL-18 (FIG. 3(f)) levels in wild-type, Asc−/−, and Nlrp3−/− mice were measured by ELISA using standard techniques on days 9 and 17 after EAE induction. IL-1β production was greater in splenocytes isolated from wild-type than Asc−/− mice 9 days after EAE induction. Cells were treated with Ultrapure LPS (100 ng/ml) alone for 24 hours, and secreted IL-1β was measured in the cell culture media using standard techniques. See FIG. 3(g). This result indicates that NLRP3 inflammasome is already activated in this condition on day 9.

Detection of active caspase-1 (casp-1 p20) in splenocytes isolated from wild-type, Asc−/−, and Nlrp3−/− mice nine days after EAE induction revealed that more activated caspase-1 was present in wild-type mice. Caspase-1 p20 and β-actin were detected in 24-hr total splenocyte culture supernatants and cell lysates, respectively, by western blot using standard techniques. Caspase-1 p20 levels were normalized to total Rac1 using NIH Image/Image J software. IFNAR signaling suppresses NLRP3-inflammasome activity in vivo. FIG. 3(i) shows serum IL-1β levels in wild-type mice 9 days after EAE induction. The left panel shows comparison of serum IL-1β levels in mice with or without IFNβ-1b treatment. IFNβ-1b (0.3×10⁵ unit/mouse) was i.p. treated at every other day from day 0 to day 8 after EAE induction. The right panel shows comparison of serum IL-1β levels between wild-type and Ifnar1−/− mice, where wild-type mice exhibited lower serum FIG. 3(j) shows IL-1β production by LPS-stimulated splenocytes isolated from mice 9 days after EAE induction. The left panel shows a comparison between wild-type mice treated with or without IFN-β-1b. The right panel shows IL-1β production by splenocytes isolated from wild-type and Ifnar1−/− mice 9 days after EAE induction. Cells were treated with Ultrapure LPS (100 ng/ml) alone for 24 hours. FIG. 3(k) shows active caspase-1 (casp-1 p20) detection in splenocytes isolated from wild-type mice with or without IFNβ-1b treatment (left panels), and from WT or Ifnar−/− mice (right panels). Spleens were harvested nine days after EAE induction, and total protein extracts from the isolated splenocytes were prepared and probed for p20 expression by western blot using standard techniques. The results show that IFN-β treatment decreased active caspase-1 p20 levels in wild-type splenocytes, and that Ifnar1−/− splenocytes had higher levels of p20 than wild-type cells.

### Example 4

### IFN-β is Effective Only for NLRP3-Dependent EAE

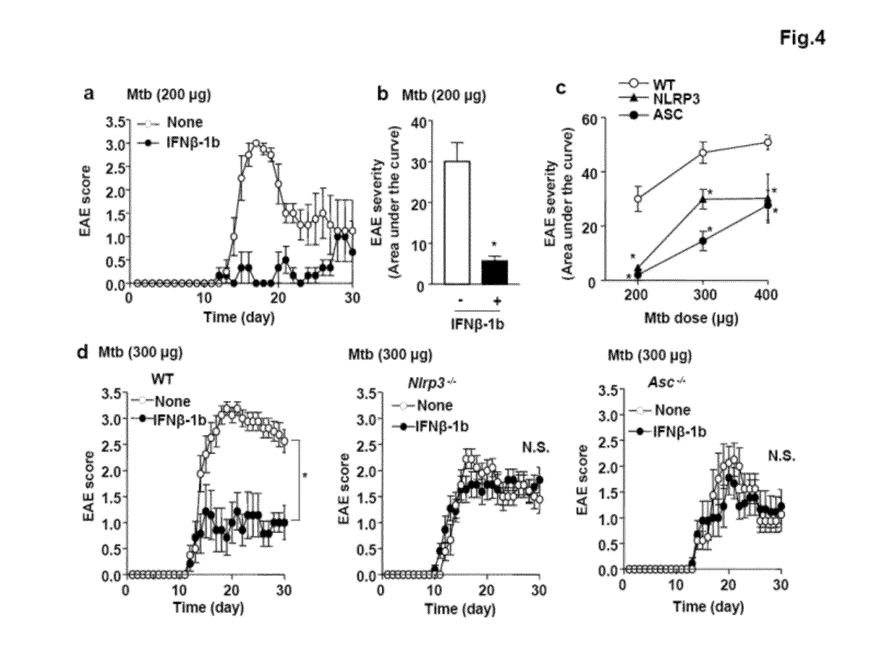

To evaluate the efficacy of IFNβ on NLRP3 inflammasome-independent EAE, we induced EAE in mice lacking a component of the NLRP3 inflammasome (Asc−/− mice and Nlrp3−/−mice) by using high dosages of Mtb as an immunization adjuvant (FIG. 4c). IFNβ successfully ameliorated EAE in wild-type mice, but not in Asc−/− and Nlrp3−/−mice (FIG. 4d). The result strongly suggested that IFNβ treatment is effective only when EAE progression depends on the NLRP3 inflammasome.

FIG. 4(a) depicts EAE development in wild-type mice with or without IFN-β-1b treatment. IFNβ-1b (0.3×10⁵ unit/mouse) was i.p. injected on every other day from day 0 to 10. For EAE induction, MOG35-55 peptide (100 μg/mouse) was emulsified with CFA (100 μl/mouse, including 200 μg of heat-killed Mycobacteria (Mtb)), and subcutaneously injected in the flanks of mice on day 0. EAE severity was scored according to standard metrics of clinical disease. FIG. 4(b) shows EAE severity evaluated by the area under the curve (AUC) from time course data shown in FIG. 4(a). FIG. 4(c) compares severity of EAE in wild-type, Asc−/−, and Nlrp3−/− mice with titrated dosages (200, 300 and 400 μg) of Mtb used for immunization. EAE severity was evaluated by AUC from time course data up to day 30. FIG. 4(d) shows that IFN-β-1b ameliorated EAE in wild-type mice induced using 300 μg Mtb (left panel), but EAE was not ameliorated in Nlrp3−/− (middle panel) and Asc−/− mice (right panel) treated with the same induction procedure. IFN-β-1b (0.3×10⁵ unit/mouse) was administered by i.p. injection every other day from day 0 to day 10 after EAE induction.

### Example 5

### Induction of NLRP3 Inflammasome-Independent EAE in WT Mice

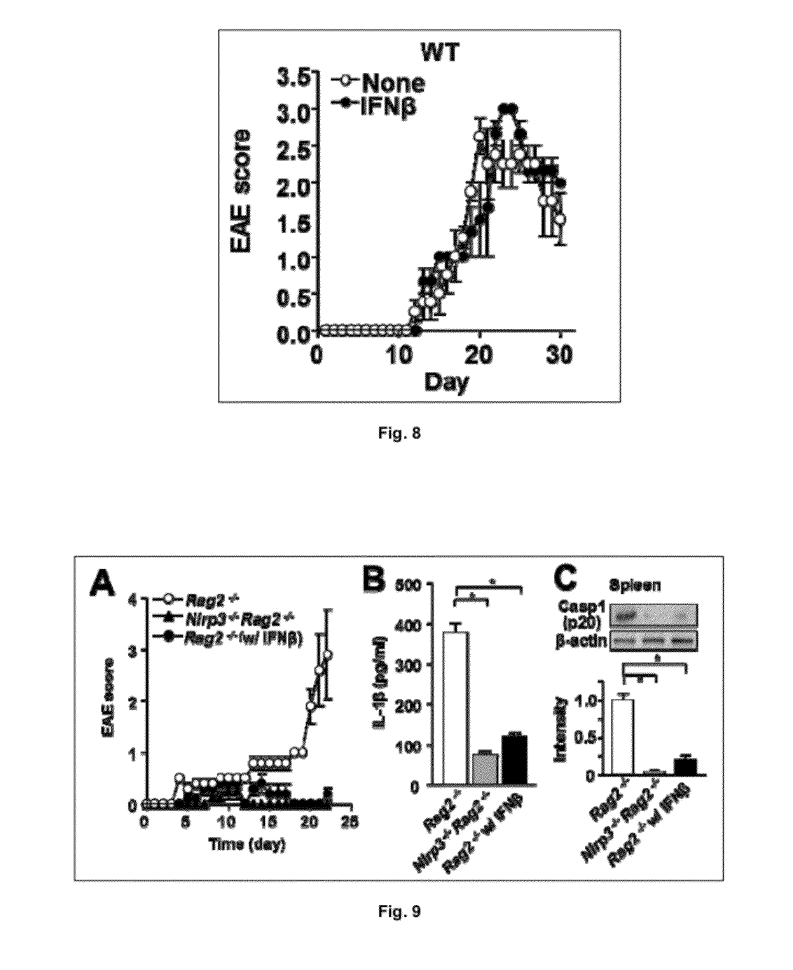

In Example 4, we showed that EAE could be induced in NLRP3 inflammasome-deficient mice (i.e., Nlrp3−/− and Asc−/− mice), and that such NLRP3 inflammasome-independent EAE cannot be treated with IFNβ. However, deficiency of NLRP3 inflammasome is rare in humans and most of MS patients are speculated to have a functional NLRP3 inflammasome. By using wild-type (WT) mice, which are NLRP3 inflammasome-sufficient, we have demonstrated that NLRP3 inflammasome-independent EAE can still be induced and that IFNβ treatment does not work (see FIG. 8).

To induce NLRP3-independent EAE in WT mice, the immunization regimen was aggressive. EAE was induced by repeated MOG immunization on day 0 and day 7. MOG35-55 peptide (125 μg/mouse) was emulsified with CFA (including 400 μg Mtb), and subcutaneously injected in the flanks of mice on day 0 and 7. Mice were also intraperitoneally injected with Pertussis toxin (200 ng/200 μL PBS/mouse) on days 0, 2 and 7. IL-1β was not detected in the EAE mice (data not shown), suggesting that the WT mice had NLRP3-independent EAE.

FIG. 8 shows that NLRP3-sufficient mice can develop NLRP3-independent EAE and that IFNβ treatment does not work when EAE is induced by an aggressive disease induction regimen. However, in MS, such artificial disease induction is not involved. The MS/human equivalent of aggressive disease induction in EAE/mouse may be induced by acute and intense microbial infections. In other words, MS patients who experienced severe infections may trigger flare-up of MS without activating the NLRP3 inflammasome. The disease history of patients with infections, in addition to the absence of NLRP3 inflammasome activity, may be a strong indication of possible IFNβ non-responders.

To investigate this possibility, EAE experiments will be performed to induce NLRP3-independent EAE in WT mice by infections. In addition, the correlation of NLRP3 activity, responsiveness to IFNβ, and history of infections, will be examined by using blood samples from MS patients.

### Example 6

### Activation of NLRP3 Inflammasome Without the Use of an Adjuvant

In general for EAE induction methods, MOG antigen is injected together with adjuvants (e.g., heat-killed Mycobacteria, or Mtb, as in the previous examples), which are usually obtained from microbes. Adjuvants stimulate innate immune cells to trigger whole immune responses. We have shown that increased dosages of Mtb shifts the character of EAE from NLRP3-dependent to NLRP3-independent. In FIG. 8, for example, twice more Mtb was used in EAE induction compared to the amount used in Example 4.

It is possible that MS is not induced as EAE by using Mtb, and the EAE induction method using adjuvant does not mimic real situations in humans (except for microbial infections, by which microbes themselves work as adjuvants). Therefore, we determined whether the NLRP3 inflammasome is activated and IFNβ therapy is effective when EAE is induced without using adjuvant. EAE was induced by adoptively transferring CD4+ T cells from WT mice, which developed EAE, into Rag2−/− (O; no T/B cells) or Nlrp−/−3 Rag2−/− (▴; no T/B cells, no NLRP3 inflammasome) recipient mice (FIG. 9A). Another group of Rag2−/− had IFNβ treatment (). Error bars indicate mean±SEM from n=5.

Evaluation of the NLRP3 inflammasome activation status was performed (see FIG. 9B). Mature IL-1β from splenic macrophages (23 days after T cell transfer) were detected after stimulation with Ultrapure LPS, which induces supply of IL-1β precursor for the NLRP3 inflammasome, which processes the precursor to generate mature IL-1β. The NLRP3 inflammasome activation status was also evaluated by detecting the activated form of caspase-1 (p20) by Western blot (FIG. 9C).

As shown in FIG. 9, without using adjuvant, EAE was induced in mice (indicated as Rag2−/− in FIG. 9) by transferring CD4+ T cells from EAE mice. This type of EAE was successfully treated by IFNβ (FIG. 9A), because the mice demonstrated the activity of the NLRP3 inflammasome (FIGS. 9B and 9C), judging from IL-1β production and activation of caspase-1. FIG. 9 also demonstrated that EAE was not induced under the condition when the T cell transfer recipients were lacking NLRP3. We confirmed that IFNβ suppresses NLRP3 inflammasome activity by showing that IFNβ treated mice demonstrated the suppression of NLRP3 inflammasome activity (FIGS. 9B and 9C).

### Example 7

### Inflammatory T Cells are Recruited into Brains, Rather than Spinal Cord, in NLRP3-Independent EAE

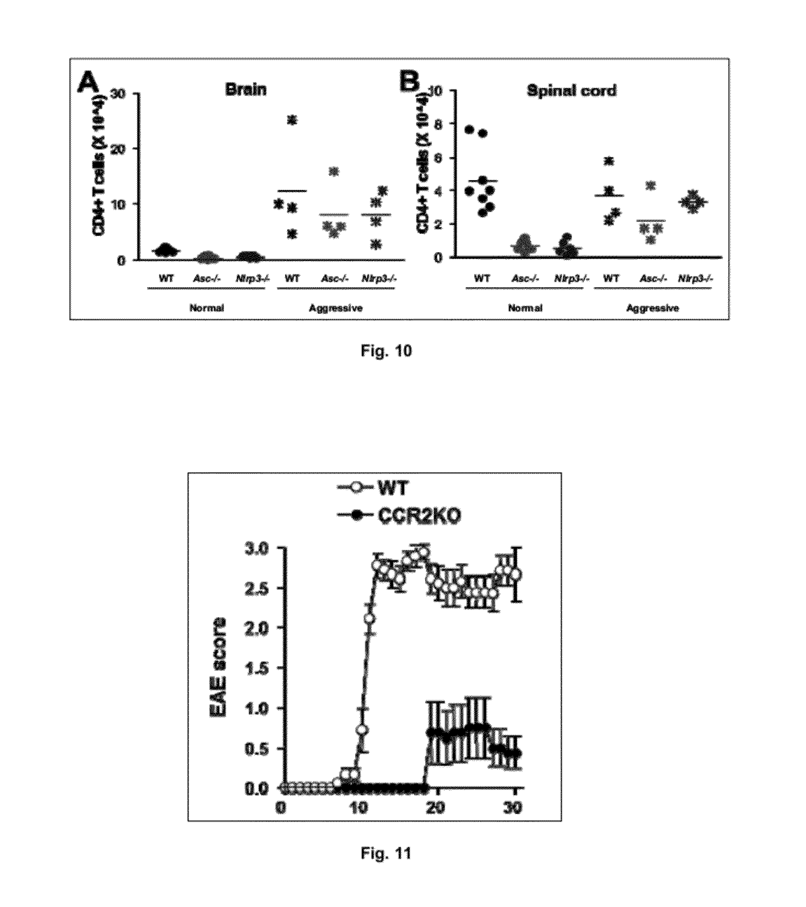

CD4+ T cell infiltration into the CNS was investigated in mice with NLRP3-dependent EAE (i.e., mice with “normal” disease induction, as described in Example 4) compared to mice with NLRP3-independent EAE (i.e., mice with “aggressive” disease induction, as described in Example 5). CD4+ T cell numbers were analyzed in brains (FIG. 10A) and spinal cords (FIG. 10B) of mice that develop EAE at the peak of EAE (“Aggressive”) compared to mice with normal disease induction (“Normal”). In the normal EAE induction, MOG35-55 peptide (100 μg/mouse) was emulsified with CFA (100 μl/mouse, including 200 μg of Mtb), and subcutaneously injected in the flanks of mice on day 0. Mice were also intraperitoneally injected with Pertussis toxin (200 ng/200 μl PBS/mouse) on days 0 and 2. In the aggressive EAE, MOG35-55 peptide (125 μg/mouse) was emulsified with CFA (100 μl/mouse, including 400 μg of Mtb), and subcutaneously injected in the flanks of mice on day 0 and 7. Mice were also intraperitoneally injected with Pertussis toxin (200 ng/200 μl PBS/mouse) on days 0, 2, and 7. Mice were harvested to evaluate the cell infiltration in the brain and spinal cords around the time of the disease peak.

FIG. 10A shows that a critical phenotype of NLRP3-independent EAE was massive inflammatory T cell infiltration in the brain (FIG. 10A; Results shown with asterisks; Horizontal bars show mean values.). With normal disease induction regimen (shown with circles), which induces NLRP3-dependent EAE, T cells largely infiltrates into spinal cord (FIG. 10B), rather than the brain (FIG. 10A).

Clinical data indicated that IFNβ therapy does not work for MS patients who also develop optic neuritis, in which inflammatory cells infiltrate into optic nerve (data not shown). Considering that proximity of optic nerve and the brain, it is expected that MS associated with optic neuritis in a patient can be used to classify or identify the patient as having, or having an increased likelihood of having or developing, the NLRP3-independent subtype of MS.

### Example 8

### Targeting CCR2 to Treat NLRP3-Independent EAE/MS

We performed a series of gene expression analyses using microarray, RT-PCR-array and standard RT-PCR analyses for screening various genes and found that a chemokine receptor, CCR2, plays a role in the induction of NLRP3-independent EAE, providing for a new class of therapeutics for patients who are non-responsive to IFNβ therapy.

NLRP3-independent EAE was induced by the aggressive EAE induction regimen (described in Example 5) in WT and Ccr2−/− mice. The disease score was monitored daily in the WT (◯) and Ccr2−/− mice () (FIG. 11). The aggressive EAE induction regimen, which makes WT mice develop NLRP3-independent EAE, failed to induce EAE in CCR2-deficient mice suggesting that CCR2 deficient mice are resistant to EAE by aggressive disease induction. From the results it is expected that functional blockade of CCR2 will ameliorate or at least provide reduction in the symptoms or severity of NLRP3-independent EAE. Thus, as the data and description provided herein establishes NLRP3-independent EAE as a model of IFNβ non-responsive MS, these results identify CCR2 inhibitors, such as are known in the art, as a useful class of therapeutics for the treatment of NLRP3-independent and IFNβ-untreatable MS.

### Example 9

### IFNβ Inhibits NLRP3 Inflammasome Activity by Suppressing ROS Generation by Mitochondria

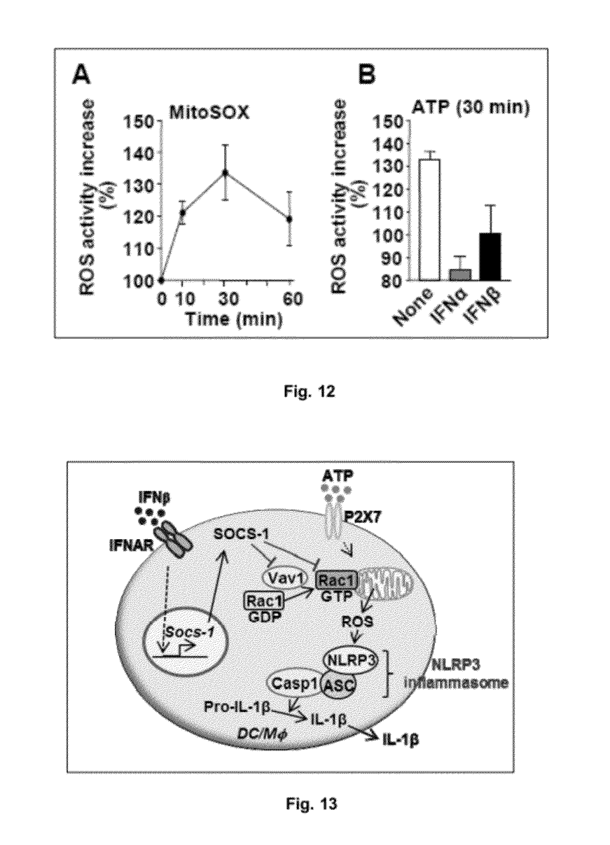

Example 2 showed that IFNβ suppresses ROS generated by NADPH oxidase. This example assessed whether ROS generated by the mitochondria may play a role in NLRP3 inflammasome activation. We tested whether IFNβ suppresses ROS generated by mitochondria with MITOSOX™ Red (Invitrogen™). A time course of ATP-induced mitochondrial ROS generation in macrophages was generated using macrophages incubated with 5 mM ATP for the time indicated in FIG. 12A. Macrophages were incubated with 5 mM ATP for indicated time at 37° C. in complete RPMI medium. ROS activity increase was calculated as mean fluorescence intensity value increase (percent) of MITOSOX™ RED staining by flow cytometry. An increase of mean fluorescent intensity (MFI) of the MITOSOX™ staining is shown in FIG. 12(A, B) as the reference point of MFI at time=0 (i.e., no ATP treatment). The ROS activity increase was calculated as mean fluorescence intensity value increase (percent) of MITOSOX™ Red staining by flow cytometry (FIG. 12A).

Suppression of mitochondrial ROS generation by type-1 IFNs was examined. Cells were incubated with 5 mM ATP for 30 min rIFNα or rIFNβ (1,000 units/mL) was added 24 hr prior to the ATP treatment. Cells were incubated with 5 mM ATP for 30 min rIFNα or rIFNβ (1,000 units/ml) was added 24 hr prior to the ATP treatment. As shown in FIG. 12B, mitochondrial ROS was suppressed both by IFNα and IFNβ.

We claim:

1. A method for identifying a patient with an autoimmune disease as non-responsive to treatment with a drug, the method comprising:
a. obtaining a sample from the patient; and
b. determining an expression level of a marker in the sample, wherein the patient is identified as non-responsive to treatment with the drug when the expression level of the marker in the sample is not increased relative to a control expression level, wherein the marker indicates NLRP3 inflammasome activity.

2. The method of claim 1, wherein the marker is IL-1β and/or IL-18.

3. The method of claim 1, wherein the marker is caspase-1.

4. The method of claim 1, wherein the drug comprises an inhibitor of NLRP3 inflammasome activity.

5. The method of claim 4, wherein the drug comprises interferon-β.

6. The method of claim 1, wherein the autoimmune disease is multiple sclerosis.

7. The method of claim 1, wherein the sample comprises blood.

8. The method of claim 1, wherein the sample comprises cerebrospinal fluid.

9. The method of claim 1, further comprising processing the sample to substantially purify a cell type in the sample.

10. The method of claim 9, wherein the cell type is a monocyte.

11. The method of claim 1, further comprising reviewing the medical history of the patient for presence of microbial infections and/or optic neuritis.

12. The method of claim 1, further comprising measuring T cell infiltration in the brain and spinal cord.

13. A method of treating an NLRP3-independent autoimmune disease in a subject, the method comprising administering an effective amount of a CCR2 inhibitor to the subject.

14. A kit comprising:
at least one reagent capable of specifically binding to a marker of NLRP3 inflammasome activity to quantify the amount of the marker in a sample from a subject;
and
a buffer.

15. The kit of claim 14, wherein the at least one reagent comprises an oligonucleotide or an antibody.

16. The kit of claim 14, wherein the marker of NLRP3 inflammasome activity is IL-1β and/or IL-18.
